(19) United States
(12) Patent Application Publication
(10) Pub. No.: US 2010/0330187 A1
(43) Pub. Date: Dec. 30, 2010

(54) CAPSICUM FOOD ADDITIVE AND USES THEREOF

(75) Inventors: David Bravo, Yverdon les Bains (CH); Sergio Calsamiglia, Sant Cugat del Valles (ES)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: PANCOSMA SOCIETE ANONYME POUR L'INDUSTRIE DES PRODUITS BIOCHIMIQUES, Le Grand-Saconnex (CH)

(21) Appl. No.: 12/811,775

(22) PCT Filed: Jan. 16, 2009
(86) PCT No.: PCT/IB2009/000095

§ 371 Date: Aug. 31, 2010

(30) Foreign Application Priority Data
Jan. 23, 2008 (FR) 08.00341

(51) Int. Cl. A61K 9/14; A61K 31/16; A61P 1/00

(52) U.S. Cl. 424/490; 514/627

(57) ABSTRACT

The invention relates to a food additive that includes, relative to the total weight thereof: about 3.5 wt % of capsicum oleoresin containing 6 wt % of a capsaicine and dihydrocapsaicine mixture; about 5.5 wt % of cinnamaldehyde; about 9.5 wt % of eugenol; the balance up to 100% consisting of hydrogenated vegetable oils. The invention also relates to the uses of said additive for improving the daily distribution of food ingestion by animals, for increasing the amount of water drunk by animals, or for preparing a food product intended for the preventive or therapeutic treatment of animal digestive disorders, such as acidosis or bloating. This food additive is particularly adapted for ruminants such as bovine cattle.

The invention relates to a food additive comprising capsicum oleoresin. Such a food additive can, in particular, be added to the food of certain animals, such as bovine cattle, with a view in particular to distributing their food intake during the day or else to preventing digestive disorders.

### BACKGROUND OF THE INVENTION

Capsicums constitute a genus of plants which can be distinguished by the presence of oleoresin containing alkaloids, capsaicin and dihydrocapsaicin, generally in their fruits. These alkaloids are known to cause irritation and a heat sensation when they are ingested.

### BRIEF DESCRIPTION OF THE INVENTION

The inventors have discovered that, despite the irritation and the heat sensation that it causes, capsicum oleoresin, which contains a mixture of capsaicin and dihydrocapsaicin, can have beneficial effects when it is used as a food additive.

The subject of the invention is therefore a food additive comprising capsicum oleoresin.

Other features and advantages of the invention will now be described in detail in the description which follows and which is given with reference to the attached figures which represent:

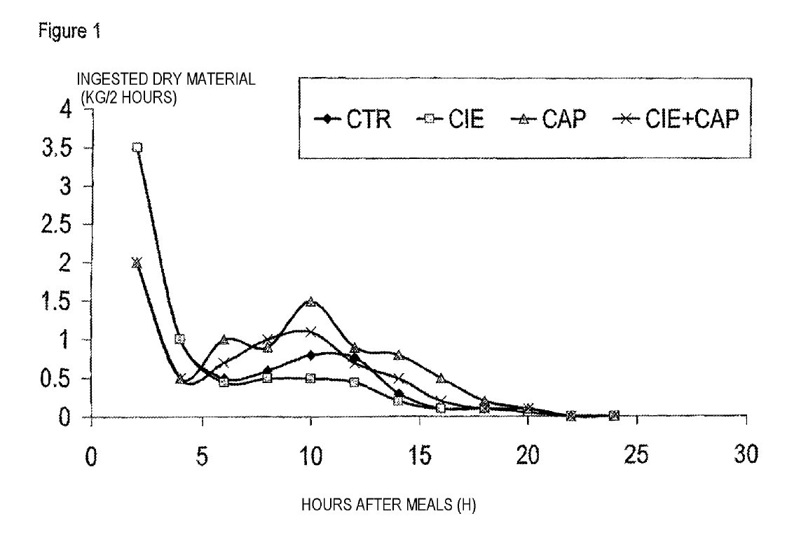

FIG. 1: curves representing the amount of food ingested by heifers as a function of the time elapsed since the food was made available; and

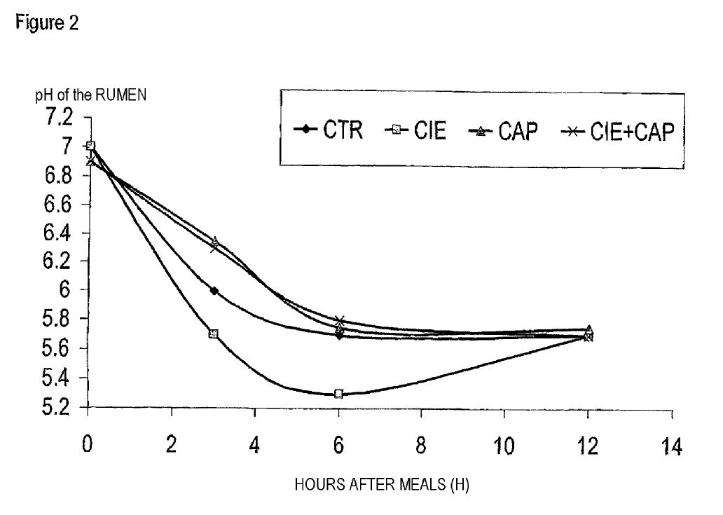

FIG. 2: change in the pH measured in the rumen of heifers as a function of the time elapsed since ingestion of the food.

### DETAILED DESCRIPTION OF THE INVENTION

The term “capsicum oleoresin” should be understood to mean the oleoresin originating in general from the fruit of a capsicum such as Capsicum baccatum, Capsicum baccatum var. pendulum, Capsicum annuum, Capsicum chinense, Capsicum frutescens and Capsicum pubescens.

The amount of mixture of capsaicin and dihydrocapsaicin present in the food additive according to the invention generally contains between 4 and 15 wt %.

The food additive according to the invention can advantageously also contain cinnamaldehyde (trans-cinnamaldehyde, of empirical formula C₉H₃O) and/or eugenol (4-allyl-2-methoxyphenol, of empirical formula C₁₀H₁₂O₂).

By way of example, mention may be made of the food additive containing, relative to the total weight thereof:
about 3.5 wt % of capsicum oleoresin, this oleo-resin containing about 6 wt % of a mixture of capsaicin and dihydrocapsaicin relative to the total weight thereof;
about 5.5 wt % of cinnamaldehyde; and
about 9.5 wt % of eugenol;
the balance up to 100% being constituted of hydrogenated vegetable oils.

The food additive is generally in the form of a powder which is generally constituted of particles having a size that can range from 90 μm to 1000 μm.

The particles can advantageously be completely encapsulated by an external encapsulating layer. The encapsulation thus limits the irritant effect of the capsicum. In addition, it makes it possible to obtain microspheres that can release the oleoresin on different sites of the digestive tract, depending on the desired objective, by virtue of suitable adaptation of the parameters of the method of encapsulation. Such a method of encapsulation is described in the French patent application filed under No. 06 55035 (FR20060055035).

When it is used to feed animals, the food additive according to the invention can be added to an animal feed concentrate. Such a feed concentrate is well known to those skilled in the art and can comprise soya hulls, corn grain, cakes, by-products from wheat or corn ethanol production, etc.

In the field of animal farming, with animals such as bovine cattle, goats, sheep, pigs, ducks, geese and rabbits, the food additive according to the invention can be added to the fodder, such as grass, alfalfa, hay, etc.

The food additive may also be used to prepare an animal feed comprising an animal feed concentrate, fodder and the food additive in question.

As an example of animal feeds, mention may be made of that constituted of:
0 to 50 parts by weight of feed concentrate;
0 to 50 parts by weight of fodder; and
5×10⁻⁵ to 2×10⁻⁴ part by weight of additive according to the invention.

The animal feed may be prepared according to a method comprising a step of preparing a mixture comprising the feed concentrate, the fodder and the food additive. These constituents can be mixed in any order.

The food additive according to the invention can be used to more successfully spread out the food ingestion by animals during the day.

The food additive according to the invention can also be used to increase the amount of water drunk by animals.

It can also be used to prepare a feed intended for the preventive or curative treatment of animal digestive disorders, in particular acidosis and bloating.

The food additive according to the invention is particularly suitable for ruminants, especially bovine cattle, and most especially heifers.

### EXAMPLES

### Animals Tested

The food additive according to the invention was tested on four Holstein heifers having an average initial live weight of 360 kg. These heifers were given a 1 centimeter canula. They were used in a 4×4 latin square. Each of the four periods lasted 3 weeks, one week of adaptation, one week of recording consumption and one week of sampling the ruminal fluid. The heifers were individually housed in connecting stalls.

### Diets

The animals were fed once a day at 8 a.m. The ration was constituted of 90% feed concentrate and 10% barley straw distributed ad libitum, to 110% of the consumption of the previous day. If the consumption changed, the amount offered was adjusted. The concentrate composition is given in detail in table 1.

TABLE 1
Food composition of the concentrate (expressed in parts by weight)

| Starting material | Content |
|---|---|
| Barley, grain | 32.2 |
| Corn, grain | 27.9 |
| Soya, cakes | 13.3 |
| Soya, hulls | 8.1 |
| Wheat | 7.5 |
| Spent corn grain | 7.2 |
| Sunflower cakes | 2.8 |
| Vegetable fat | 1.1 |
| Calcium carbonate | 0.5 |
| Dicalcium phosphate | 0.5 |
| Vitamin supplements | 0.4 |

The ration was constituted, on a dry material basis, of 16.1% of crude proteins, 22.0% of plant walls (NDF, Neutral Detergent Fiber) and 54.3% of non structural carbohydrates (NSC). It was formulated to meet or exceed the requirements recommended by the NRC (2001) for a Holstein heifer weighing 360 kg and showing a daily growth of 1.15 kg/day.

### Food Additives Tested

The following food additives were used:
CAP: a food additive according to the invention constituted of 2.75 g of capsicum oleoresin (of Capsicum frutescens) containing 6 wt % of a mixture of capsaicin and dihydrocapsaicin;
CIE: a food additive which serves as a food additive for comparison and which is constituted of 3.24 g of a mixture containing 17 wt % of cinnamaldehyde and 28 wt % of eugenol, the balance up to 100% being constituted of hydrogenated vegetable oils;
CAP+CIE: 5.99 g of a food additive according to the invention constituted of a mixture of CAP (2.75 g) and CIE (3.24 g) as defined above.

The food additives were added manually to the food made available daily.

### Experimental Measurements

The measurements were carried out in the following way: a first control group of heifers (control, abbreviated to “CTR”) did not receive any food additive, a second group received the CAP food additive, the third group received CIE and the fourth received CAP+CIE.

On the fifth day of the week, the animals were moved from the individual stall to the experimental barn. The consumptions were recorded after 3 days of adaptation.

An automated system was used to record the ration consumption starting from day 9 to day 14 of each experimental period.

Troughs with a 120 l capacity were mounted on a leakproof digital balance for each stall. Iron bars were placed between the heifers and the balance in order to prevent the animals from putting their feet or head thereon. Each balance was programmed to transmit the weight of the food each minute. This period was chosen because it is considered to be a satisfactory indicator of short-term feeding behavior. The information was downloaded onto a computer.

Each observation of feed weight was classified as an “eating” observation when the food ingested (the real weight of food minus the previous weight) was greater than 10 grams, or “unstable” when the measurement was recorded while the head of the animal was pressing on the balance while eating. Otherwise, the observation was classed as “non eating”.

The data was corrected when the heifers pressed on the balance. The consumption was calculated by multiplying the disappearance of feed from the balance by the dry material content of the feed. The dry material content of the feed concentrate and of the straw was determined.

The residues separated from the straw and from the feed concentrate were weighed and the dry material content was determined. The residues were separated using a fodder separator (Penn State University, Pennsylvania, United States) and the particles of less than 8 millimeters were considered to be the concentrate.

The dry material content was determined by drying in an oven at 105° C. for 24 h. The feeding behavior was determined by calculating the consumption per 2-hour period. The consumption of water was verified using individual drinking troughs.

The pH of the rumen was measured by sampling the ruminal fluid using a trocar. The pH of the rumen was measured immediately with a portable pH meter. The pH was determined in the morning, just before feeding and at 3, 6 and 12 h after feeding. It was measured for the first 3 days of the third week.

### Statistics

The statistical analysis employed the generalized linear model using the GLM procedure of the SAS software.

In order to determine the effect of the treatments on the following parameters: daily water consumption, daily feed consumption, pH, the following model was employed:

Y=μ+Pᵢ+Jⱼ+Alₖ+Tₗ+εᵢⱼₖₗₘ

with:
P=experimental period with i=1 to 4
A=heifer with k=1 to 4
J=day of each period with j=1 to 3
T=experimental treatment with l=1 to 4 (with CAP and CIE and CIE+CAP and CTR for control).

For the analysis of the feed consumption curve, the following model was used:

Y=μ+Pᵢ+l−2hₘ+Aₖ+Tₗ+εᵢⱼₖᵢₘ

with:
l−2hₘ=the ingestion of dry material during the previous 2h
P=experimental period with i=1 to 4
A=heifer with k=1 to 4
J=day of each period with j=1 to 3
T=experimental treatment with l=1 to 4 (with CAP and CIE and CIE+CAP and CTR).

It was considered that the residual error obeyed a normal law. The analysis of the differences between the means was carried out using the Tukey test.

### Results

### a) Effects on Feed and Water Consumption

The results are indicated in table 2 in the form of amounts of dry material ingested and of water drunk.

The daily ingestion of dry material (9.3 kg) did not differ between the experimental treatments. The heifers receiving the CAP treatment consumed the least, numerically (8.6 kg/day against 9.6 kg/day against 9.7 kg/day against 9.4 kg/day, respectively, for the CTR, CIE and CIE+CAP treatments). The consumptions of feed concentrate and of straw were not influenced by the treatments.

TABLE 2
Effects of the CAP and CIE treatments, alone or in combination,
on the consumption of feed concentrate, of straw and of water

| | | | | | | Statistical value P (1) | | | |
|---|---|---|---|---|---|---|---|---|---|
| | | Treatments | | | | CAP | CIE | CIE + | CIE |
| | | | | | CIE + | vs. | vs. | CAP vs. | vs. |
| | | CTR | CAP | CIE | CAP | CTR | CTR | CTR | CAP |
| Dry | Mean | 9.6 | 8.6 | 9.7 | 9.4 | NS | NS | NS | NS |
| material | | | | | | | | | |
| ingested | | | | | | | | | |
| (kg/day) | | | | | | | | | |
| Straw | Mean | 0.8 | 0.6 | 0.8 | 0.8 | NS | NS | NS | NS |
| ingested | | | | | | | | | |
| (kg/day) | | | | | | | | | |
| Feed | Mean | 8.8 | 8.0 | 8.9 | 8.7 | NS | NS | NS | NS |
| concentrate | | | | | | | | | |
| ingested | | | | | | | | | |
| (kg/day) | | | | | | | | | |
| Water drunk | Mean | 31.9 | 37.5 | 35.5 | 38.0 | 0.038 | NS | 0.013 | NS |
| (L/day) | | | | | | | | | |

(1): “vs” = compared with (“versus” in Latin)
NS = the difference between the two means is not significant (the statistical value P is greater than 10%).
** = the difference between the two means is significant (the statistical value P is less than 5%).

When the heifers receive the CIE mixture, a slight increase in water consumption was recorded (35.5 L/day against 31.9 L/day). When the heifers received capsicum alone or in combination with the CIE mixture, the water consumption increased: 37.5 and 38.0 L/day against 31.9 L/day, respectively, for capsicum alone or with CIE.

### b) Effects on Feeding Behavior

TABLE 3
Effects of the CAP and CIE treatments, alone
or in combination, on feeding behavior

| | | | | | Statistical value P | | | | |
|---|---|---|---|---|---|---|---|---|---|
| Hours | Treatments | | | | CAP | CIE | CIE + | CIE + | CIE |
| after | | | | CIE + | vs. | vs. | CAP vs. | CAP vs. | vs. |
| meals | CTR | CAP | CIE | CAP | CTR | CTR | CTR | CIE | CAP |
| From 1 to 2 | 3.23 | 2.03 | 3.17 | 2.09 | ** | NS | 0.007 | ** | NS |
| hours | | | | | | | | | |
| From 3 to 4 | 0.68 | 0.64 | 0.95 | 0.65 | NS | NS | NS | NS | NS |
| hours | | | | | | | | | |
| From 5 to 6 | 0.54 | 0.86 | 0.46 | 0.73 | NS | NS | NS | NS | 0.04 |
| hours | | | | | | | | | |
| From 7 to 8 | 0.58 | 0.82 | 0.57 | 1.01 | NS | NS | *** | *** | NS |
| hours | | | | | | | | | |
| From 9 to | 0.89 | 1.33 | 0.67 | 1.06 | NS | NS | NS | NS | *** |
| 10 hours | | | | | | | | | |
| From 11 to | 0.71 | 0.79 | 0.61 | 02.77 | NS | NS | NS | NS | NS |
| 12 hours | | | | | | | | | |
| Total in kg | 6.63 | 6.47 | 6.43 | 6.31 | | | | | |

(1): “vs” = compared with (“versus” in Latin)
NS = the difference between the two means is not significant (the statistical value P is greater than 10%).
** = the difference between the two means is highly significant (the statistical value P is less than 5%, but greater than 1%).
*** = the difference between the two means is very highly significant (the statistical value P is less than 1%).

When the heifers received the CTR feed, most of the ingestion took place within the first 2 hours following the meal, with 3.23 kg of feed consumed out of a total of 6.63 kg consumed in all. The heifers then reduce their consumption until 9-10 hours after the meal, and carry out a second peak of consumption with a meal of 0.89 kg (out of 6.63 kg in total).

When the heifers were fed with a CIE mixture, they did not modify their feeding behavior.

When the heifers are fed with the CAP treatment, they reduce their feed consumption during the first two hours after the meal (2.03 kg versus 3.23 kg). During the next 4 hours, the feed consumption is the same between the groups. Seven hours after the meal, the heifers receiving the CAP treatment gradually increase their consumption: 0.82 kg versus 0.58 kg from 7 to 8 hours, then 1.33 kg against 0.89 kg from 9 to 10 hours after the meal.

The addition of capsicum to the feed containing the CIE mixture (i.e. the CIE+CAP treatment) reduces the feed consumption during the first 2 hours after the meal (2.09 against 3.23 kg). Then, 7 hours after the meal, the heifers receiving the capsicum and the CIE mixture simultaneously increase their feed consumption like the heifers receiving only the CAP treatment.

TABLE 4
Effects of the CAP and CIE treatments, alone or in combination, on the average
rate of consumption per 30 minutes, the maximum consumption per 30 minutes
and the time spent consuming feed for the various experimental treatments

| | | | | | | CAP | CIE | CIE + | CIE + |
|---|---|---|---|---|---|---|---|---|---|
| | | | | | CIE + | vs. | vs. | CAP vs. | CAP vs. |
| | | CTR | CAP | CIE | CAP | CTR | CTR | CTR | CIE |
| Mean rate of | Mean | 1.8 | 1.4 | 2.0 | 1.8 | NS | NS | NS | NS |
| consumption | | | | | | | | | |
| per 30 minutes | | | | | | | | | |
| Maximum | Mean | 4.8 | 2.8 | 4.7 | 2.6 | | | | |
| consumption | | | | | | | | | |
| per 30 minutes | | | | | | | | | |
| Time spent | Mean | 8.7 | 12.4 | 7.2 | 10.1 | * | NS | * | NS |
| consuming feed | | | | | | | | | |

(1): “vs” = compared with (“versus” in Latin)
NS = the difference between the two means is not significant (the statistical value P is greater than 10%).
* = the difference between the two means is significant (the statistical value P is less than 10%, but greater than 5%).

As indicated in table 4, the mean consumption during a period of 30 minutes does not differ between the treatments. The time spent consuming feed is modified by the treatments. There is no difference when the control and CIE diets are compared, whereas the addition of capsicum to the feed increases the time spent consuming feed. This effect is recorded when the capsicum is consumed alone (12.4% against 8.7%) or when it is added to the CIE mixture (10.1% against 8.7%).

The maximum consumption per 30 minutes is also modified by the treatments. The CIE mixture does not modify this criterion (4.7 kg versus 4.8 kg, P), whereas it is decreased in the two treatments containing capsicum oleoresin: 2.8 kg and 2.6 kg, respectively, for CAP or CAP+CIE.

### b) Effects on the pH of the Rumen of the Animals

The pH of the rumen decreases starting from 3 hours after the meal. The greatest reduction occurs for CIE and tends to result in a pH which is below that of the other treatments and of the control; after 6 hours, the pH is below that measured with CAP and CIE+CAP, cf. FIG. 2. No significant difference is recorded between the other pH values.

### c) Interpretation of the Results

The results clearly show that the addition of capsicum to a ruminant growth diet modifies the feeding behavior. The capsicum greatly reduces, overall, the size of the first meal. In addition, the capsicum increases the size of the meal taken around 9-10 hours after the food has been made available.

In addition, the capsicum significantly reduces the amount of food ingested during 30 minutes.

The addition of capsicum alone or as a mixture with eugenol and cinnamaldehyde reduces the size of the first meal (table 3).

It is clearly apparent, according to FIG. 1, that the CAP additive, alone or in combination with CIE, causes a decrease in the starting peak and a better daily distribution of food consumption (2nd peak 9-10 hours after the starting peak). By way of comparison, it is observed that CIE does not show a second peak.

Thus, by limiting the size of the first meal, the capsicum reduces digestive disorders, in particular the risk of acidosis and/or of bloating.

Furthermore, the CAP additive, with or without CIE, causes a significant increase in water consumption (cf. table 2).

1-16. (canceled)

17. A food additive comprising capsicum oleoresin, cinnamaldehyde and eugenol, for use in improving the daily distribution of feed ingestion by animals.

18. A food additive comprising capsicum oleoresin, cinnamaldehyde and eugenol, for use in increasing the daily amount of water drunk by animals.

19. The food additive as claimed in claim 17, in which the capsicum oleoresin contains between 4 and 15 wt % of a mixture of capsaicin and dihydrocapsaicin.

20. The food additive as claimed in claim 18, in which the capsicum oleoresin contains between 4 and 15 wt % of a mixture of capsaicin and dihydrocapsaicin.

21. A food additive, comprising, relative to the total weight thereof:
about 3.5 wt % of capsicum oleoresin containing 6 wt % of a mixture of capsaicin and dihydrocapsaicin;
about 5.5 wt % of cinnamaldehyde;
about 9.5 wt % of eugenol; and
the balance up to 100% being constituted of hydrogenated vegetable oils.

22. The food additive as claimed in claim 20, wherein the food additive is in the form of a powder.

23. The food additive as claimed in claim 21, wherein particles constituting the powder comprise an external encapsulating layer.

24. An animal feed concentrate, containing a food additive as claimed in claim 20.

25. A fodder, containing a food additive as claimed in claim 20.

26. An animal feed comprising an animal feed concentrate, fodder and a food additive as claimed in claim 20.

27. The animal feed as claimed in claim 26, comprising:
0 to 50 parts by weight of feed concentrate;
0 to 50 parts by weight of fodder; and
5×10⁻⁵ to 2×10⁻⁴ part by weight of food additive.

28. A method for preparing an animal feed as claimed in claim 26, comprising mixing the feed concentrate, the fodder and the food additive in any order.

29. A method of preventive treatment of animal digestive disorders, comprising administering a food additive as claimed in claim 21 to an animal as preventive treatment of animal digestive disorders.

30. A method of curative treatment of animal digestive disorders, comprising administering a food additive as claimed in claim 21 to an animal as curative treatment of animal digestive disorders.

31. The method as claimed in claim 29, wherein the digestive disorders are acidosis.

32. The method as claimed in claim 30, wherein the digestive disorders are acidosis.

33. The method as claimed in claim 29, wherein the digestive disorders are bloating.

34. The method as claimed in claim 30, wherein the digestive disorders are bloating.

35. The method as claimed in claim 30, wherein the animals are ruminants.

36. The method as claimed in claim 35, wherein the ruminants are bovine cattle.
